(19) United States
(12) Patent Application Publication
(10) Pub. No.: US 2021/0361164 A1
(43) Pub. Date: Nov. 25, 2021

(54) MEDICAL BIOSENSOR DEVICE, SYSTEM, AND METHOD

(71) Applicant: Ses Development, LLC, Richardson, TX (US)

(72) Inventor: Joseph Bogdan, Fairview, TX (US)

(21) Appl. No.: 17/256,982

(22) PCT Filed: Jun. 28, 2019
(86) PCT No.: PCT/US2019/039795

§ 371 Date: Dec. 29, 2020

Related U.S. Application Data
(60) Provisional application No. 62/692,448, filed on Jun. 29, 2018.

(51) Int. Cl. A61B 5/00; H04W 4/38; G16H 40/67; A61B 5/257; A61B 5/308

(52) U.S. Cl. CPC A61B 5/0006; H04W 4/38; G16H 40/67; A61B 2562/0209; A61B 5/002; A61B 5/308; A61B 5/746; A61B 5/257

(57) ABSTRACT

A communication system including a biosensor device that includes a frame configured to contain leads for electrode patches, which may comprise attachment points for electrode patches configured to allow signals detected by electrode patches attached to the attachment points to transmit to a processing unit, and configured to space the patches apart for use in medical monitoring. The frame may also be configured to contain one or more batteries and to electrically connect such batteries to the processing unit. The system may also include a smart phone or other mobile device configured to wirelessly receive data from the biosensor device and configured to transmit the received data to at least one of a cloud-based processing system, a database, and a healthcare provider's office.

### CROSS-REFERENCE TO RELATED APPLICATIONS

This application is a filing under 35 U.S.C. 371 of International Application No. PCT/US2019/039795 filed Jun. 28, 2019, entitled “Medical Biosensor Device, System, and Method,” which claims priority to U.S. Provisional Patent Application No. 62/692,448 filed Jun. 29, 2018 by Joseph Bogdan and entitled “Medical Biosensor Lead” which is incorporated herein by reference as if reproduced in its entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Healthcare providers may wish to remotely monitor a patient during their day-to-day activity and collect data about the health of the patient. Wearable monitoring devices may be worn by patients, where the wearable monitoring devices may be configured to collect biometric data over the course of the time the patient is wearing the device and may be configured to ultimately communicate this information to the patient's healthcare provider, thereby contributing to the healthcare providers assessment of the health of the patient.

### SUMMARY

In an embodiment, a biosensor device comprising a frame configured to contain leads for electrode patches, comprising attachment points for electrode patches configured to allow signals detected by electrode patches attached to the attachment points to transmit to a processing unit, and configured to space the patches apart for use in medical monitoring, wherein the frame is also configured to contain one or more batteries and to electrically connect such batteries to the processing unit.

In an embodiment, a communication system comprising a biosensor device comprising a frame configured to contain leads for electrode patches, which may comprise attachment points for electrode patches configured to allow signals detected by electrode patches attached to the attachment points to transmit to a processing unit, and configured to space the patches apart for use in medical monitoring, wherein the frame may also be configured to contain one or more batteries and to electrically connect such batteries to the processing unit; and a smart phone or other mobile device configured to wirelessly receive data from the biosensor device and configured to transmit the received data to at least one of a cloud-based processing system, a database, and a healthcare provider's office.

In an embodiment, a method of using a biosensor device comprising attaching one or more standard electrode patches to a frame of the biosensor device, attaching a processing unit to the frame, wherein the processing unit is configured to receive data from the one or more standard electrode patches, removably attaching the biosensor device to the patient's skin via the one or more standard electrode patches, continuously detecting patient data via the one or more standard electrode patches, transmitting, by the processing unit, the detected data via short-range wireless communication to a mobile device located in proximity to the biosensor device, retransmitting, by the mobile device, the detected data via a longer-range wireless to at least one of a cloud processing system, a remote medical database, a healthcare provider office, analyzing all detected data remotely from the biosensor device by comparing the detected data to one or more profiles indicative of various alerts, and transmitting an alert to at least one of the wireless device and the biosensor device in response to the detected data matching one or more such profiles.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a top view of a medical biosensor device, according to an embodiment of the disclosure.

FIG. 2 illustrates an alternative view of the medical biosensor device of FIG. 1 in which a processing unit has been removed, according to an embodiment of the disclosure.

FIG. 3 illustrates a bottom view of a medical biosensor device, according to an embodiment of the disclosure.

FIG. 4 illustrates a communication system according to an embodiment of the disclosure.

FIG. 5 illustrates an electronics high-level block diagram according to an embodiment of the disclosure.

FIG. 6 illustrates wearable software architecture according to an embodiment of the disclosure.

FIG. 7 illustrates a modular system for a medical biosensor device according to an embodiment of the disclosure.

FIG. 8 illustrates an exploded view of the modular system for a medical biosensor device of FIG. 7 according to an embodiment of the disclosure.

FIG. 9 illustrates another modular system for a medical biosensor device according to an embodiment of the disclosure.

FIG. 10 illustrates a top view of yet another modular system for a medical biosensor device according to an embodiment of the disclosure.

FIG. 11 illustrates a bottom view of the modular system for a medical biosensor device of FIG. 10 according to an embodiment of the disclosure.

FIG. 12 illustrates a top view of yet another modular system for a medical biosensor device according to an embodiment of the disclosure.

FIG. 13 illustrates a bottom view of the modular system for a medical biosensor device of FIG. 12 according to an embodiment of the disclosure.

FIG. 14 illustrates a lead configuration for use with the modular system for a medical biosensor device of FIG. 12 according to an embodiment of the disclosure.

FIG. 15 illustrates another lead configuration for use with the modular system for a medical biosensor device of FIG. 12 according to an embodiment of the disclosure.

FIG. 16 illustrates another view of a medical biosensor device of FIG. 12 according to an embodiment of the disclosure.

FIG. 17 illustrates another view of a medical biosensor device of FIG. 12 according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definitions of terms shall apply throughout the application.

The term “comprising” means including but not limited to and should be interpreted in the manner it is typically used in the patent context.

The phrases “in one embodiment,” “according to one embodiment,” and the like generally mean that the particular feature, element, component, structure, or characteristic following the phrase may be included in at least one embodiment of the presently disclosed subject matter, and may be included in more than one embodiment of the presently disclosed subject matter (importantly, such phrases do not necessarily refer to the same embodiment).

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field (for example +/−10%); and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Disclosed herein are embodiments of medical biosensor devices of the type that might be worn by a patient/user to monitor vital signs, signals, parameters, other health-related information, An example of such a medical biosensor devices might be a device utilized by a patient/user to detect, gather, analyze, and/or transmit electrocardiogram (ECG) information of the patient (e.g., to allow for more effective analysis of a patient's heart condition). Typically, one or more patches on or associated with the device interface with the user's skin, allowing electrical activity (such as a patient's heartbeat, for example) within the patient's body to be detected.

Typically, conventional wearable ambulatory medical devices (such as biomedical biosensors) often may have at least two challenges which can negatively impact use. The first is the need to provide an affordable, stable interface with the patient to collect data without artifact (i.e., by removing outliers and errors), and the second is the need for extended battery life sufficient to allow the patient the freedom to carry on with daily activities unconstrained while wearing the device. Typically, conventional wearable cardiac monitoring devices tend to require substantial power in order to miniaturize such devices to a compact size convenient for wearable medical monitoring.

Typically, conventional medical biosensor devices often have one or more problems with the weight of such devices, the power supply for the device, and/or the cost for the electrodes of the device. For instance, existing ambulatory cardiac monitors are often powered by rechargeable lithium-ion batteries, for example, because of the cost of replacement batteries. Rechargeable lithium-ion batteries are heavy, thus creating a power-to-weight ratio design challenge. Due to this challenge, such monitoring devices typically have to have batteries replaced and/or recharged 1, 2, or even more times a day, which reduces patient compliance in wearing the device.

Conventional medical biosensor devices employ only a single battery, for example, to reduce weight, to supply power to capture ECG data to onboard memory and process the data onboard, for example, using various algorithms to detect arrhythmias or other abnormalities. Conventional medical biosensor devices employ such algorithms based upon the premise that such onboard processing might reduce power consumption of the such devices by reducing the amount of data being transmitted (for example, to aid in solving the power-to-weight issue discussed above). However, the use of such algorithms also means that such devices do not transmit all patient ECG information; that is, such conventional devices do not transmit beat-to-beat ECG data. Instead, these medical biosensor devices typically only transmit data (e.g., ECG beats) predetermined to be abnormal based on the limited and/or simplified onboard algorithms (or processing) embedded in the device. Furthermore, such conventional devices typically have a lithium-ion battery located within the processing unit, for example, integral within the processing unit such that the processing unit might be removed from the electrode leads and plugged into a wall socket for recharging. Patch technologies are becoming more popular but also have usage limitations because of the cost of manufacturing of proprietary patch designs on a relatively small scale.

The disclosed embodiments of medical biosensor devices overcome one or more of the issues associated with such conventional wearable ambulatory medical monitoring devices. The medical biosensor devices disclosed herein can be worn completely concealed under the user's clothing, and may have the ability to collect and transmit beat-to-beat ECG data continuously (e.g., 24-hours per day) for substantially longer than such conventional devices, for example, at least 20 days, or at least 30 days, or at least 45 days (for example, without recharging and/or replacing batteries), while using standard electrode patches.

In some embodiments, the disclosed medical biosensor devices may have minimal (or even no) onboard memory, for example, no storage of the patient's detected information/data (such as ECG data) on the device) and no onboard algorithms/processing (e.g., no analysis of the detected patient information/data on the device itself). Instead, the medical biosensor devices disclosed herein may use a wireless communication module (e.g., a low-energy Bluetooth (BLE) module) to transmit encrypted ECG data to a cellular or other mobile device (such as a smart phone or computer) which, in turn, may be configured to transmit the data onward to a cloud server, an external database, and/or a doctor's office (e.g., for automatic algorithms (for example, analysis by a computer operating algorithm(s)) and/or other analysis to detect any issues and/or issue/transmit warnings to the device (for example, instructing the patient to go to the hospital, call 911, call their doctor, etc.)). The cloud server, etc. may then analyze and/or store the data (e.g., within the cloud or other memory), thus reducing the amount of power needed at the device itself compared to most standard medical monitors.

In some embodiments, the medical biosensor devices disclosed herein may comprise a housing/frame configured to house one or more high-capacity cell-batteries (e.g., similar to watch batteries). The high-capacity cell-batteries batteries may power the device for a minimum of 30-days (e.g., 30-50 days) without needing to be recharged and/or replaced.

In some embodiments, the housing/frame may also be configured to house a processing unit (e.g., device motherboard), which may be removably attached to the housing/frame, for example, so that the housing/frame (which may include the batteries) may be used in a disposable manner (along with the electrode patches), while the processing unit may be reused. In other embodiments, the processing unit may be permanently affixed (e.g., non-removably attached) to the housing/frame. In some embodiments, the batteries may be permanently disposed within the housing/frame, while in other embodiments the housing/frame may be configured to allow the batteries to be replaced (e.g., to allow easy removal and replacement of batteries once their power is waning/spent). Additionally, in some embodiments additional batteries may be located on the housing/frame apart from (e.g., external to) the processing unit.

In some embodiments, processing unit may be configured to cause the medical monitoring device to power “on” automatically only upon connection to the patient and/or to automatically power off if/when removed from the patient. Thus, in some embodiments, no on-off (e.g., power) switch may need to be included.

In some embodiments, the housing/frame may be configured to provide an electrical connection between the processing unit and one or more patches configured to interface with the user/patient. For example, the housing/frame may include one or more electrical leads extending between the processing unit and an attachment point for the patches. The electrical leads may be contained within a housing/frame, to protect them and/or to keep them from entanglements. In some embodiments, the housing/frame may be configured for use with two, three, four, or more electrode patches. In some embodiments, traditional/standard electrode patches may be used. In comparison to the custom electrode patches needed for many medical biosensor devices, traditional/standard electrode patches may be used, which may be obtained/produced at a much lower cost and with much greater availability. With capitated reimbursement from Medicare and private insurances, it can be vital to keep cost under control to ensure a user is able to afford to use the device as instructed by a healthcare provider.

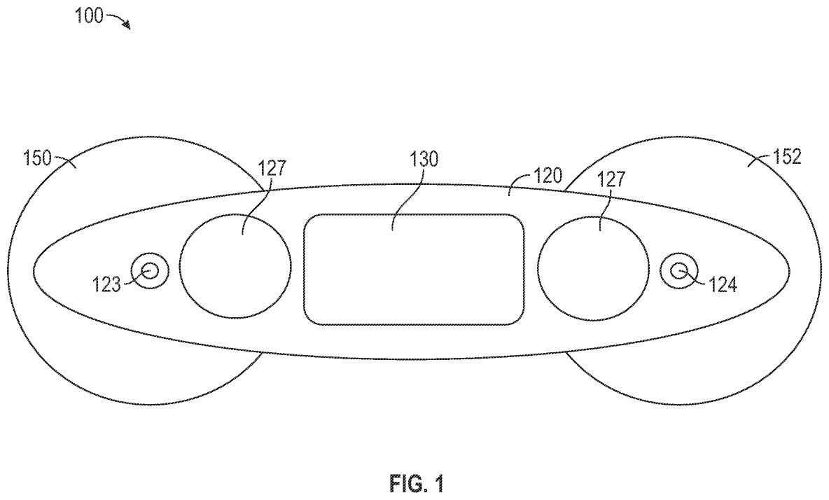

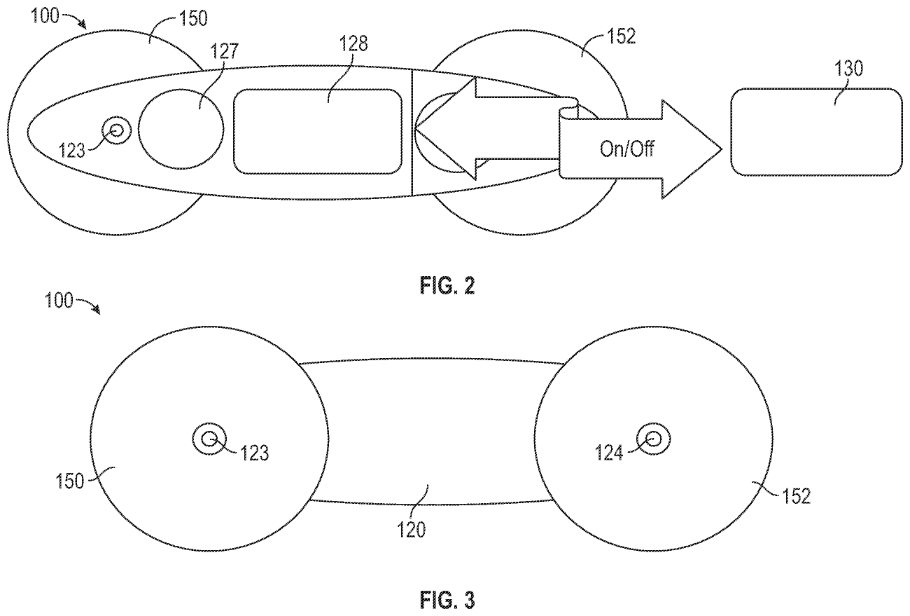

Referring now to FIGS. 1-3, an embodiment of a medical biosensor device 100, such as an ECG biosensor device, is shown. The device 100 may comprise a housing/frame 120, which in this embodiment may be ovaloid in shape (for example, having a smooth exterior surface to prevent interference with a user's clothing or other wearables). The housing/frame 120 may be configured for mounting and/or enclosing one or more of the processing unit 130, one or more batteries 127 (for example, two high-capacity cell-batteries (e.g., similar to watch batteries)), and/or one or more electrode patches 150 and 152 (for example, two three, or four patches). The electrode patches 150 and 152 may be standard patches, as opposed to custom and/or proprietary patches, although a proprietary patch may be used in some embodiments. The electrical leads may be configured to electrically connect the patches 150 and 152 when mounted on the frame 120 (for example, when attached via patch-lead attachment points 123 and 124) to the processing unit 130 when the processing unit 130 is mounted on the frame 120, for example, when the processing unit 130 is attached to the frame 120 via an interface, such as the processing unit interface 128. The electrical leads may be entirely contained within and/or mounted on the frame 120, for example, so that there are no loose electrical leads. Thus, the electrical leads typically electrically connect the patch-lead attachment points 123 and 124 (and thereby the patches 150 and 152 when mounted thereon) to the processing unit 130 and/or processing unit attachment point 128.

In some embodiments, the device 100 is configured such that activation might occur automatically when the device 100 is mounted (via the patches 150 and 152) to the patient in a way that completes the circuit between the patches 150 and 152, the processing unit 130, and the batteries 127 (e.g., so that current flows through the patient to complete the circuit). The batteries 127 (e.g., the electrical contacts/terminals for the battery compartment(s)) may be electrically connected to the processing unit 130 in order to power the device 100 for detection of the patient's ECG data and allow continuous detection and transmittal of the data for at least 30 days (e.g., 30-50 days). In some embodiments, the batteries 127, for example, two high-capacity cell-batteries (e.g., similar to watch batteries), may be permanently attached to and/or disposed within the frame 120; alternatively, the batteries may be removably attached to and/or disposed within the frame 120, for example, within a battery compartment(s) within the frame 120. In some embodiments, the device 100 could be viewed as such a frame 120, configured in this manner for attachment of standard electrodes, batteries, and a processing unit (e.g., so that the battery powers the processing unit and the processing unit interacts with the patches to detect patient ECG data when the device is mounted on a patient's skin (e.g., via (solely) the two standard patches)).

In some embodiments, the processing unit 130 comprises a low power and/or short range wireless transmitter, such as a Bluetooth transmitter. The processing unit 130 may be configured to cause the wireless transmitter to transmit data detected by the device 100 (such as detected ECG data) to an external mobile device (such as a smart phone) located in proximity of the transmitter (e.g., within range of the Bluetooth). The processing unit may be configured to cause the data detected by the device 100 to be transmitted on a continuous basis, for example, substantially contemporaneous with the receipt of the data by the processing unit 130 (for example, in real-time or substantially in real-time).

Upon receipt of the data from the wireless transmitter of the processing unit 130, the external mobile device may then re-transmit the data over longer distances (e.g., via standard Wi-Fi and/or cellular device), for example, to a cloud for processing, storage, etc. In some embodiments, the external mobile device may be configured to re-transmit the data received from the device 100 on a continuous basis, for example, substantially contemporaneous with the receipt of the data by the processing unit 130 (for example, in real-time or substantially in real-time); alternatively, the external mobile device may be configured to re-transmit the data received from the device 100 on a batch basis, for example, to re-transmit the data for in predetermined quantities and/or at predetermined intervals, such as about every 30 seconds, about every 60 seconds, about every 90 seconds, about every 120 seconds, about every 150 seconds, about every 180 seconds, about every 240 seconds, about every 3 minutes, about every 4 minutes, about every 5 minutes, about every 6 minutes, about every 7 minutes, about every 8 minutes, or about every 10 minutes.

In some embodiments, for example, as shown in FIG. 1, the processing unit 130 may be removably attached (electrically and physically) to the frame 120, for example, via the processing unit interface 128. The processing unit interface 128 may be configured to provide physical engagement of the processing unit 130 within the frame 120, for example, such that when engaged via the processing unit interface 128, the processing unit 130 is securely retained in place with respect to the frame 120. The processing unit interface 128 may also be configured to provide electrical connection between the processing unit 130 and the electrical leads disposed on or within the frame 120, for example, via a suitable number and arrangement of electrical contacts. Alternatively, in some embodiments the processing unit 130 may be permanently attached to the frame 120, for example, such that removal from the frame 120 would require tools and/or damage the device.

In some embodiments, the device 100 includes two standard electrode patches 150 and 152 which, in some embodiments, could be permanently attached to the frame 120 or, in some embodiments, may be removably attached to the frame 120 (e.g., electrically and physically). The batteries 127 and the configuration of the device 100 would be such that the device 100 could detect and transmit patient beat-to-beat ECG continuously (e.g., non-stop, 24 hours per day) for at least 30 days (e.g., from 30-50 days) without the need to recharge or replace batteries. Not intending to be bound by theory, and contrary to conventional wisdom, by transmitting all or substantially all of the data (e.g., beat-to-beat data) captured by the processing unit 130 to the external device (as opposed to processing the data via the processing unit 130), the device 100 may exhibit substantially improved durations over which the device can be used without replacement or recharging.

In some embodiments, a system which comprises the medical biosensor device 100 and a mobile device (such as a smart phone) configured to receive the data (e.g., Bluetooth signal, with the patient's ECG data) from the processing unit 130. The mobile device could then be configured to either perform processing itself on the received data, or to retransmit the data using standard wireless, etc. for external processing at a distance (e.g., cloud-based processing, etc.). Some such systems might also include the cloud-based computing system and/or an external database and/or a doctor's office, which might be configured to perform processing (for example, in real-time or substantially in real-time, using algorithms) and/or storage of the data.

In some embodiments, the sizing and weight of the device 100 may allow for an effective attachment to a patient's skin solely using the electrode patches and/or without additional attachment and/or support mechanisms, effective ECG detection, and effective daily wear without unduly interfering with the patient's activity. Additionally, the configuration of the device 100 may help to improve patient compliance (for example, by not requiring removal and/or recharge or battery replacement for at least 30 days) while also maximizing effective detection and screening (e.g., by performing beat-to-beat ECG).

In some embodiments, a medical biosensor device, for example, as described in FIGS. 1-3, may be used in a method comprising one or more of the following steps: (removably) attaching standard electrode patches to the frame; (removably) attaching the processing unit to the frame; removably attaching the biosensor device to the patient's skin (e.g., (solely) by attaching the frame to the patient using the patches); detecting, collecting, and/or transmitting (e.g., by the processing unit) all detected ECG data (e.g., for continuous beat-to-beat ECG monitoring (e.g., 24 hours a day) for at least 30 days (e.g., 30-50 days)) without recharging and/or changing of batteries; transmitting (e.g., by the processing unit) the detected data via Bluetooth to a mobile device (such as a smart phone) located in proximity to the biosensor device (e.g., without removing the processing unit from the frame); retransmitting (e.g., by the mobile device) the detected data via longer-range wireless (e.g., Wi-Fi and/or cellular service) to cloud processing, remote medical database, doctor office, etc. (e.g., for instantaneous analysis, for example by automated algorithm); analyzing all detected data remotely (e.g., by comparing the detected ECG data to one or more profiles indicative of various medical issues/alert); transmitting (by the cloud, etc.) an alert (e.g., to the wireless device) in response to the detected data matching one or more such profiles and/or transmitting (by the wireless device) the alert to the biosensor device; switching/replacing from one type of standard patch to another standard patch in response to a patient's allergic response or other medical reason; and/or switching/replacing patches that become loose. In some embodiments, the processing unit may transmit via Bluetooth beat-to-beat ECG data (e.g., all detected data) for at least 30 (e.g., 30-50) continuous days (e.g., without recharging or replacing batteries). In some embodiments, no algorithm is used by the processing unit to analyze the data or to use or transmit less than all of the detected data and/or no ECG data is stored locally on the device (e.g., at the processing unit). In some embodiments, there are no loose lead wires (e.g., all lead wires completely contained within and/or on the frame).

Upon viewing this disclosure, persons of skill will also appreciate that the disclosed lead system embodiments can also be used in monitoring other than EGC as well (e.g., other personal medical data that could be detected via a lead system) and/or could be used for monitoring with multiple data channels (e.g., 1-12 channels of ECG data, various other data, or combinations thereof).

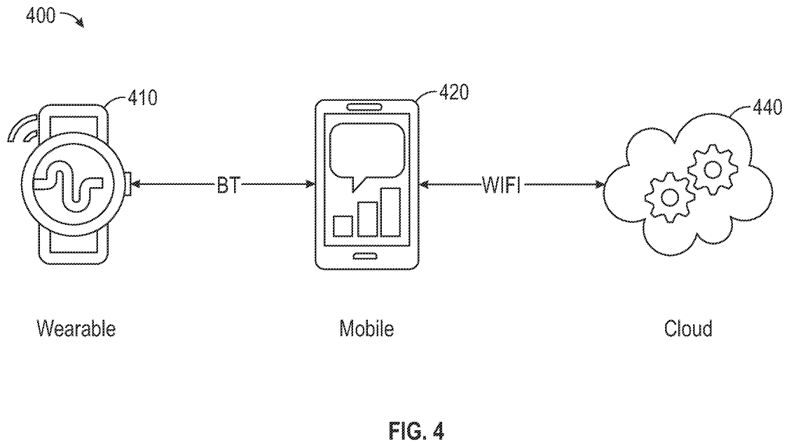

Referring to FIG. 4, a communication system 400 is shown comprising a wearable device 410 (which may be configured similarly to the medical biosensor device 100 described in FIGS. 1-3) configured to wirelessly communicate with other devices of the communication system 400. The system 400 may comprise a monitoring system that allows physicians to comprehensively monitor patients' (users') ambulatory ECG and heart rate performance. The wearable 410 may be configured to communicate with a mobile device 420 (e.g., configured to operate an application for receiving, processing, and/or transmitting data from the wearable 410), and/or a cloud database 440 which may be accessed by the user/patient and/or a healthcare provider.

In some embodiments, the wearable 410 may be configured to transmit data detected by the wearable 410 (such as detected ECG data) to an external mobile device (such as a smart phone) located in proximity of the transmitter (e.g., within range of the Bluetooth), for example, on a continuous basis, for example, substantially contemporaneous with the receipt of the data (for example, in real-time or substantially in real-time). In some alternative embodiments, the wearable 410 may be configured to buffer data for later transmission (e.g., to the communication system 400), which may be accomplished by incorporating approximately 15 minutes of storage in external memory within the device. External memory may also comprise software components, such as a real-time operating system (RTOS).

In some embodiments, the medical biosensor device 100 disclosed with respect to FIGS. 1-3 may be included within a system 400, which may further comprise a mobile device 420 (such as a smart phone) configured to receive wireless (e.g., Bluetooth) signal from the processing unit 130 of the biosensor device 100 with the patient's ECG data. The mobile device 420 may then be configured to either perform processing itself on the received data, and/or to retransmit the data using various wireless protocols for external processing at a distance (e.g., cloud-based processing, etc.). Some such systems 400 might also include the cloud-based computing system and/or an external database and/or a doctor's office, which might be configured to perform (instantaneous) processing (e.g., using algorithms) and/or storage of the data.

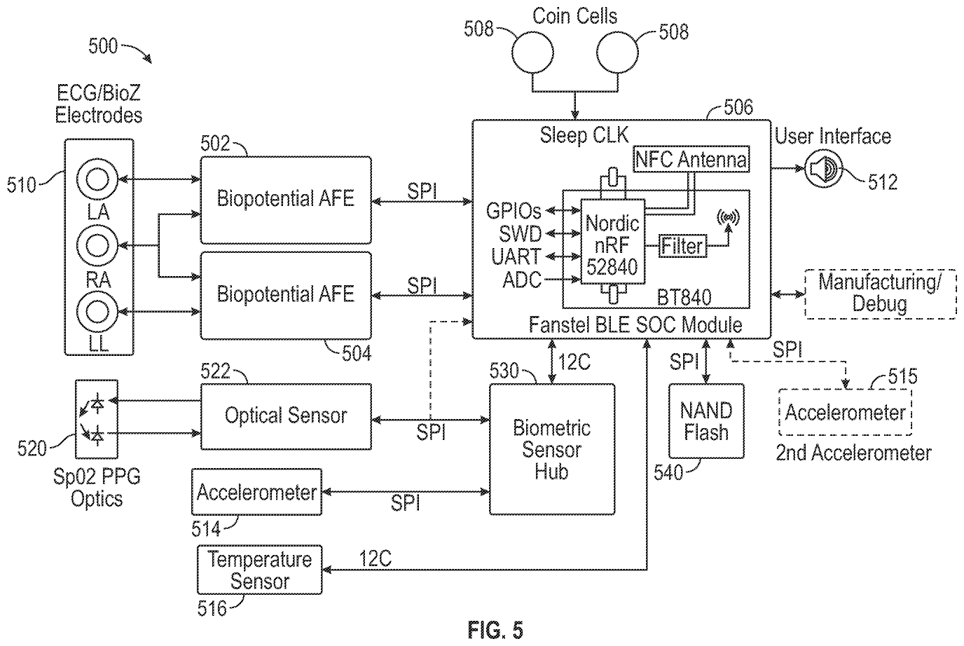

FIG. 5 illustrates an electronics high-level block diagram which may be used (e.g., incorporated into the medical biosensor device 100 of FIGS. 1-3 and/or the wearable 410 of FIG. 4. The system architecture 500 may include support for dual channel (lead) ECG 510 and for secondary features. For example, the system architecture 500 may include one or more oxygen saturation (SpO₂) sensors 520, one or more temperature sensors 514, and/or one or more accelerometers 514 and 515 that may be configured to monitor activity and/or respiratory rate. In the embodiment of FIG. 5, the system-on-a-chip (SOC) 506 is shown in a module which includes additional electronics components such as the radio antenna and includes FCC RF Certification. The system 500 may comprise one or more analog front end (AFE) modules 502 and 504 configured to receive data from the electrodes 510.

The SOC 506 may be configured to implement the computation, storage and communication functionality of the wearable. Internal memory 540 may be used to store ECG data in case transmission can be delayed. The SOC 506 may also power external systems (like the AFEs 502 and 504) from an onboard DC/DC regulator thus eliminating a separate power management integrated circuit.

The AFEs 502 and 504 may include lower power usage and performance improvements (namely having a FIFO or sample buffer) when compared to other AFEs. The AFE(s) may also include Pacer Detection and Bioimpedance measurements. The use of two or more AFEs may allow for two or more ECG channels of data. The AFEs 502 and 504 may communicate/connect with the SOC 506 via a high-speed serial peripheral interface (SPI).

In some embodiments, the SOC 506 includes a built-in DC/DC power regulator. The regulator can also power external circuitry, such as the AFE 502 and 504.

The SpO₂ sensor may be powered by a battery/power source 508 configured to generate about 5V for the illumination LEDs from 3V battery source. An exemplary non-rechargeable power source may comprise Lithium Manganese Dioxide coin cells available from multiple manufacturers. In some embodiments, the battery source may comprise disposable CR-type coin cell, based on lithium/manganese dioxide electrochemical system with a nominal voltage of 3V. As an example, the power source may comprise dual CR2450 coin cells with additional duration capacity for any “secondary” features.

The medical biosensor device 100 and/or the wearable 410 may include a user interface 512, which may comprise a haptic device or visual device (LED) to indicate power on, low battery or some other user information. First-time device turn-on can be done using a removable strip (so batteries can be inserted at time of manufacture). The medical biosensor device 100 and/or the wearable 410 can turn off after a fixed time if ECG leads are off the patient and turn back on when leads are back on. Alternatively, a small recessed button could be included to power on the device after shutdown. The user interface 512 may also comprise physical interface that may be accessed by a user for first time wearable programming. Software updates and diagnostics (for reprocessing) ideally may be done via Bluetooth to minimize any physical interfaces.

An accelerometer 514 may be used to measure patient activity, including sleep or awake state. The accelerometer 514 may comprise 3 axis support and interfaces using either SPI or I2C buses. The accelerometer may also include a temperature sensor 516 (which may be used for calibration).

In some embodiments, the memory 540 may comprise one of two types of persistent memory, for example NOR Flash and NAND Flash. Generally, NOR Flash is more expensive but is available in under 1 Gb sizes. NOR is fast for random reads but slow erase and write. NAND Flash is lower cost, but exhibits improved performance for sequential, smaller size erase blocks and better performance for erase and write.

In some embodiments, ECG data can be transmitted approximately 122 times faster than received (at 250 samples/sec, assuming BLE transmission rate of 1 Mbps, 50% transmit duty cycle or 500 ms out of 1 second, and packets are 50% data efficient). Thus, every second 512 bytes ECG are “received” from the AFE, 64,000 bytes can be transmitted wirelessly via BLE. Note that transmit data rate may be limited to lower power usage, and effective duration may be lower than calculated since buffering will be required for simultaneous receiving and transmitting of data.

Pulse oximetry (via the SpO₂ sensor(s) 520), which may also be incorporated into the medical biosensor device 100 and/or the wearable 410, may allow for non-invasive measurement of blood oxygen saturation levels, or SpO₂ (peripheral capillary oxygen saturation). In some embodiments, incorporation of SpO₂ measurement into a patch may be by a reflective (as opposed to transmissive) pulse oximetry method. In reflective pulse oximetry, LEDs illuminate the skin with a specific wavelength light and the reflected light is then measured using a photodetector for changes in absorption (also referred to as PPG or photoplethysmography). Typically, Red and IR (infrared) LEDs are used in determining PPG signals. Green LEDs have been used for heart rate detection in wrist devices.

In some embodiments, a pulse oximeter sensor may be configured for placement on the chest, for example, allowing the measurements to be taken from the core body instead of the peripheral body like finger and forehead probes. Patients with compromised peripheral blood perfusion would be able to get accurate pulse rate and SpO₂ measurements with a sensor on the chest. A chest PPG sensor is closer to the heart where oxygenated blood is pumped out of the left side of the heart. The chest may also be a good location for a reflectance PPG sensor because of the location of the sternum bone.

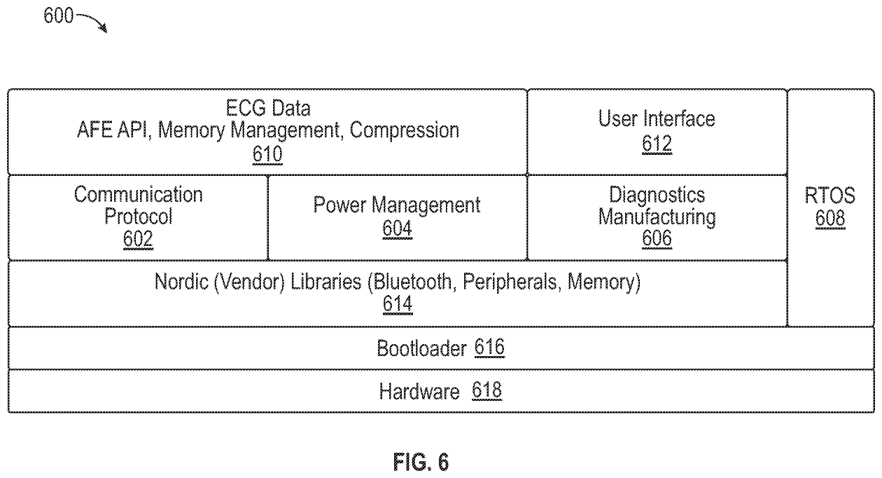

An embodiment of the software architecture is shown in FIG. 6. The ECG Data module 610 may include AFE Interface with the AFE via a SPI bus. The software will need to initialize and then receive data from the AFE. The AFE includes a 32-sample FIFO which can be set to interrupt the SOC on programmable size transfers (typically ½ of FIFO or 16-samples). At the interrupt time the SOC will remove the data, review for events such as leads-off and process the data for transmission.

The ECG Data module 610 may include memory management, where data and electrode status will be stored for later transmission via Bluetooth BLE. Storage may be short-term in a queue, or possibly in persistent flash memory for future transmission in case the mobile device cannot be reached.

The ECG Data module 610 may be configured to provide compression, which may be used to reduce the total data bandwidth and thus power used to transmit the data from the wearable to the mobile device. Typical compression rates of 2-28x have been shown for ECG13. Lossless algorithms may be considered, as well as simple adaptive PCM schemes if the integrity of the ECG can be preserved for later analysis by the operational (cloud) software.

The Communication Protocol module 602 (which may also be a wireless communication module) may be configured for communicating via Bluetooth BLE. The wearable may be configured as a peripheral with the mobile device being a central device. Initialization initializes the SOC Bluetooth radio. Advertising periodically transmits packets from the device so the mobile device can connect or bond with the wearable. Authentication certifies that wearable is bonding and communicating with a valid mobile device.

ECG Data Interface 610 may be configured to receive ECG data from the ECG Data Module and transmits using a custom upper level protocol to the mobile device. Existing BLE protocols will be used if appropriate to leverage existing BLE standard and make software less complex.

Wearable/Mobile Protocol may define the ECG data format as well as any additional wearable related data (timestamps, electrode status, battery, errors, etc.). Also included is any transmitted and received data authentication to ensure that the ECG device and communication cannot be compromised (e.g., due to a man-in-the-middle attack).

Power management module 604 may be configured to manage the wearable startup and shutdown, and the power usage of the wearable to maximize battery life. When the device is not “active” (data not available or electrodes are in leads-off state for a certain time) the SOC should be placed in deep sleep mode. Power management module 604 also monitors the state of the battery and notifies the User Interface 612 and mobile if battery is low.

In some embodiments, the software may be power balanced, for example, to reduce the peak current usage. Thus, not only is the average current but the peak current is important. CR coin cell batteries have peak currents (typically 10-15 mA), and peak current may create excessive voltage drops and result in shorter battery life.

User interface 612 May be configured to cause the wearable to directly signal the user of events and notifications such as wearable being active or battery power being low. Both visual (e.g., LED) or haptic (e.g., buzzer) devices will be considered as per user needs and power budgets.

Diagnostics module 606 may be configured to provide any power self-tests on power-on and on-demand for manufacturing. The wearable should include a persistent log for troubleshooting devices that may have failed, as well as tracking metrics (usage time, events, battery, errors) from the field.

A bootloader 616 is the SOC start-up code that initializes the system and prepares to execute the application. Included is also a means to perform an over-the-air wearable software update.

The software will leverage available software libraries available from the SOC vendor. An RTOS 608 may be required to support functionality such as managing external memory, and if needed (as determined by user needs) small footprint RTOS will be considered.

Bluetooth 614 may be configured as a continuous transmitting peripheral with data transmission equivalent to sending 256 16-bit samples per second.

In some embodiments, a single CR2450 cell could power a single channel wearable for 30 to 45 days as required, if not longer. Additional optimizations such as reduced radio power, optimized BLE transmission and lower CPU speed can extend the duration by several days while additional processing time could lower it.

The hardware 618 may include various electronic components, for example, the system architecture 500 illustrated in FIG. 5.

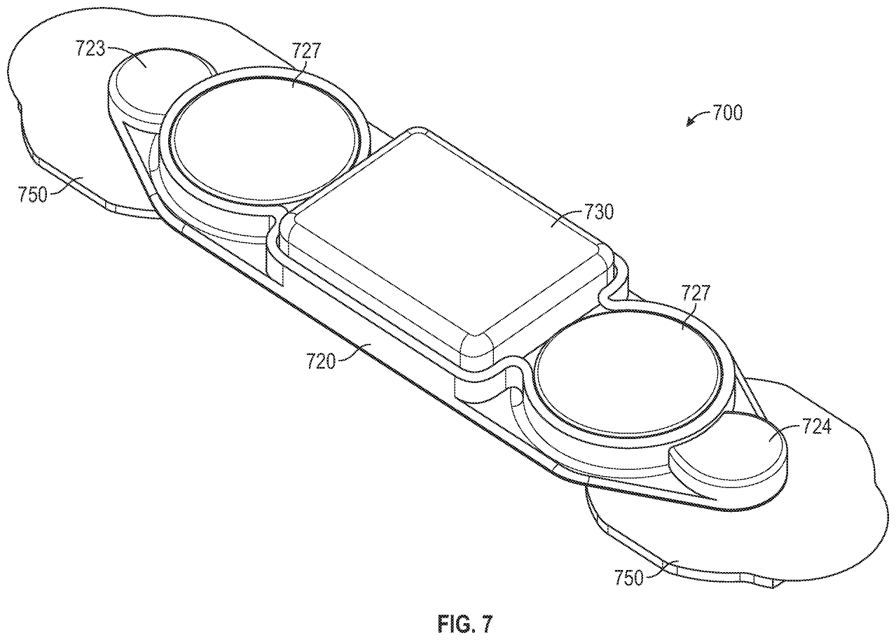

In the following embodiments, various embodiments of medical biosensor devices (e.g., wearable devices) are disclosed. In the following embodiments, for example, the embodiments of FIGS. 7-17, the various disclosed embodiments of the devices may each include a processing unit (e.g., processing unit 130), as similarly disclosed above with respect to FIGS. 1-3. In some embodiments, the processing unit (e.g., referred to as a main processing unit) may be interchangeable between the various device embodiments. For example, the same processing unit may be configured to be usable with each of the various embodiments, that is, with devices comprising various numbers of electrode patches and/or various numbers and/or configurations of channels. For example, the processing unit may be configured to detect the number of channels of the device with which the processing unit is engaged and to configure the operation of the processing unit accordingly. For example, where the processing unit receives only a single channel of data, the processing unit may configure its operation to only transmit the single channel and, where the processing unit receives multiple channels of data, the processing unit may configure its operation to transmit each of those multiple channels. Additionally, the processing unit may be configured to adjust the current supplied to the various electrode patches and/or the adjust the channels of data collected.

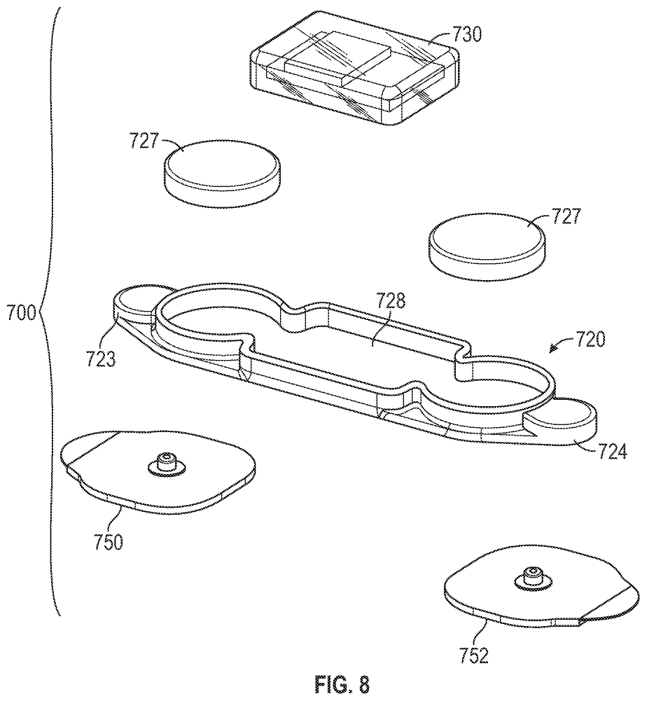

Referring to FIGS. 7-8, a modular wearable system 700 is described. FIG. 7 illustrates an assembled system/device 700 while FIG. 8 illustrates an exploded view of the system/device 700. The system/device 700 may comprise a modular design to expand the flexibility of the system. A main processing unit (MPU) 730 may be configured to connect to a platform (or frame) 720 that houses one or more batteries 727. The platform 720 may connect to one or more electrode patches 750 and 752 via patch-lead attachment points 723 and 724.

In some embodiments, a flexible platform 720 may connect the MPU 730 to the batteries 727 to allow for conforming to the curvature of different patient anatomy. In some embodiments, a flexible connecting piece (platform 720) may be significantly more comfortable than a rigid platform to wear on the chest for a sustained period and during movement.

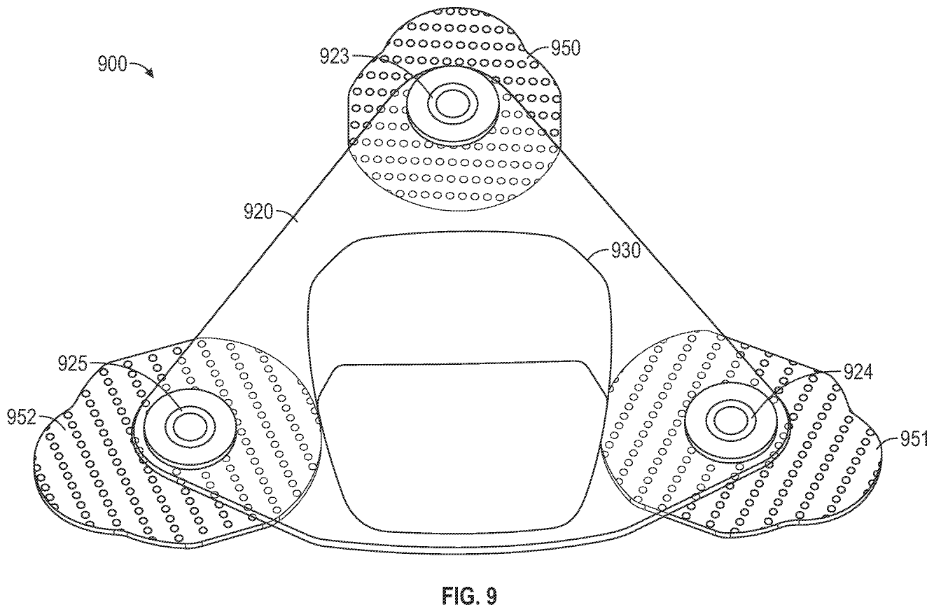

Referring to FIG. 9, some embodiments of the device may include a three electrode, two lead channel design. The device 900 may comprise a platform 920 configured to connect to an MPU 930 and configured to connect to three electrodes 950, 951, and 952 via three lead-attachment points 923, 924, and 925. In some embodiments, the electrodes 950, 951, and 952 may be spaced at a minimum of approximately 2 inches between the electrodes. In some embodiments, approximately 80 mm (or 3.15 inches) spacing between the electrodes may be desired or recommended, growing the overall footprint.

The addition of a second lead channel (when compared to the two-electrode device 100 described in FIG. 1) may provide more accurate readings for the user's biometric data, allowing a first channel to be a main data channel, while the second channel may function to confirm the data generated by the first channel and in some cases rule out artifact.

In some embodiments, a custom tool or a unique fastener may be used to remove the MPU 930 from the platform 920, thereby preventing a patient from tampering with the connection of the MPU to the rest of the assembly.

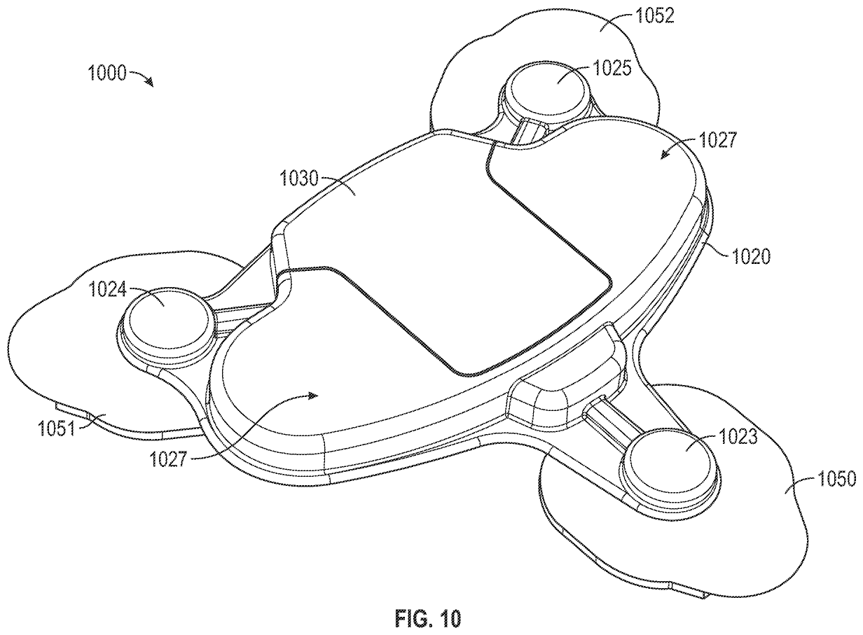

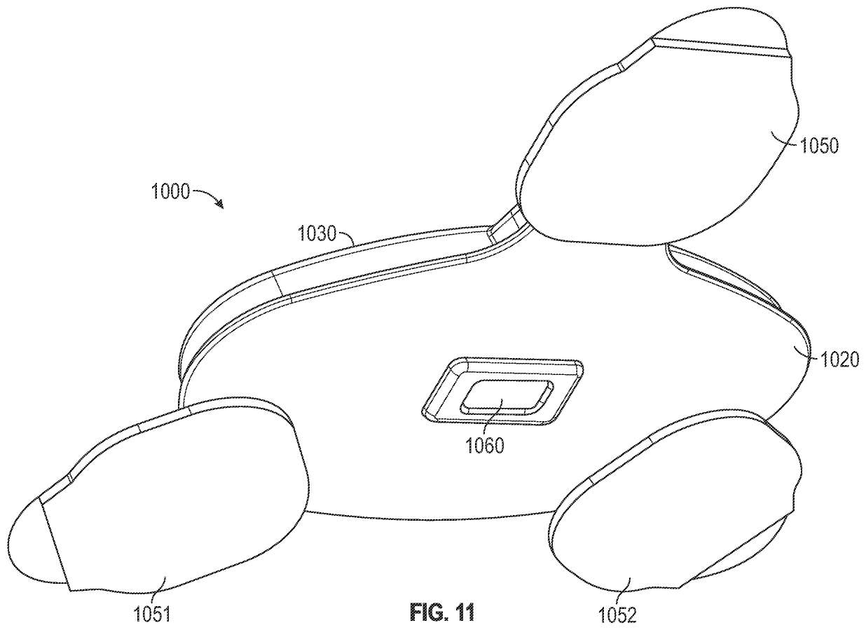

FIGS. 10 and 11 illustrate additional views of a three electrode, two lead channel device 1000, the device 1000 comprising a platform 1020 configured to connect to a MPU 1030 and configured to connect to three electrodes 1050, 1051, and 1052 via three lead-attachment points 1023, 1024, and 1025. The platform 1020 may also be configured to connect to one or more batteries (or battery compartments) 1027.

FIG. 11 illustrates an SpO₂ sensor 1060 incorporated into/attached to the platform 1020 and configured to contact the user's skin when the electrode patches 1050, 1051, 1052 are secured in place against the user's skin. The SpO₂ sensor 1060 may be positioned to avoid interference with any of the three electrodes 1050, 1051, 1052 and/or the lead connections between the three electrodes. As described above, the SpO₂ sensor 1060 may be connected to the MPU 1030 and configured to send sensor data to the MPU 1030, which may be further communicated via a wireless communication system.

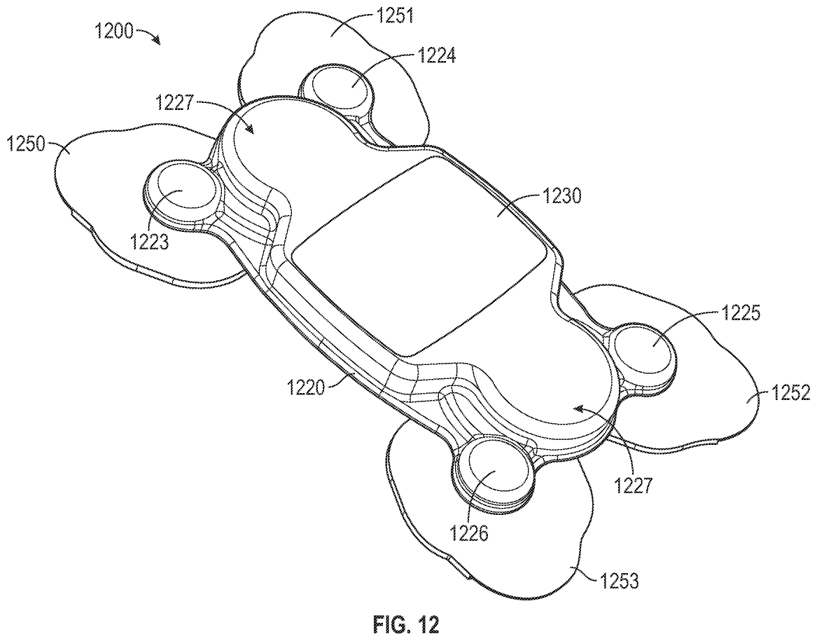

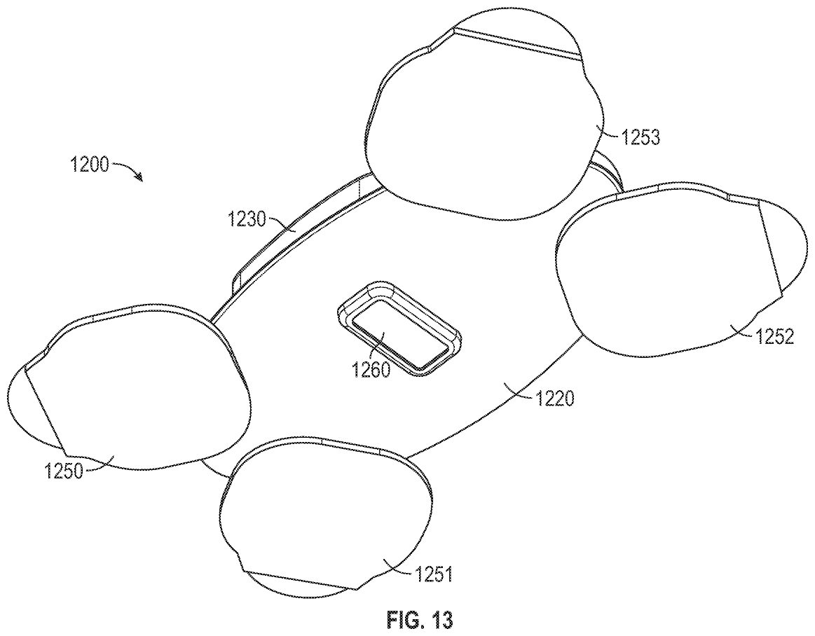

FIGS. 12 and 13 illustrates a device 1200 comprising four attachment patches 1250, 1251, 1252, and 1253. In some embodiments, only three of the patches will function as (or contain) an electrode, while the fourth patch comprises an adhesive patch. In other embodiments, all four patches will function as electrodes. In other embodiments, only two of the four patches will function as electrodes.

The device 1200 may also comprise a platform 1220 configured to connect to an MPU 1230 and configured to connect to the up to four patches 1250, 1251, 1252, and 1253 via up to four attachment points 1223, 1224, 1225, and 1226. The platform 1220 may also be configured to connect to one or more batteries (or battery compartments) 1227.

FIG. 13 illustrates an SpO₂ sensor 1260 incorporated into/attached to the platform 1220 and configured to contact the user's skin when the electrode patches 1250, 1251, 1252, and/or 1253 are secured in place against the user's skin. The SpO₂ sensor 1260 may be positioned to avoid interference with any of the electrodes 1250, 1251, 1252, and/or 1253 and/or the lead connections between the electrodes. As described above, the SpO₂ sensor 1260 may be connected to the MPU 1230 and configured to send sensor data to the MPU 1230, which may be further communicated via a wireless communication system.

In the embodiment of the device 1200 comprising three electrodes and one “dummy” patch, the additional fourth patch may serve to improve adhesion and secure the connection between the platform 1220 and the user's skin. In some embodiments, the addition of a fourth patch may change the shape and/or size of the platform 1220 to allow for a more compact overall device 1220, possibly making the device 1220 more comfortable for the user. The additional patch may also improve and secure the connection between the SpO₂ sensor 1260 and the user's skin.

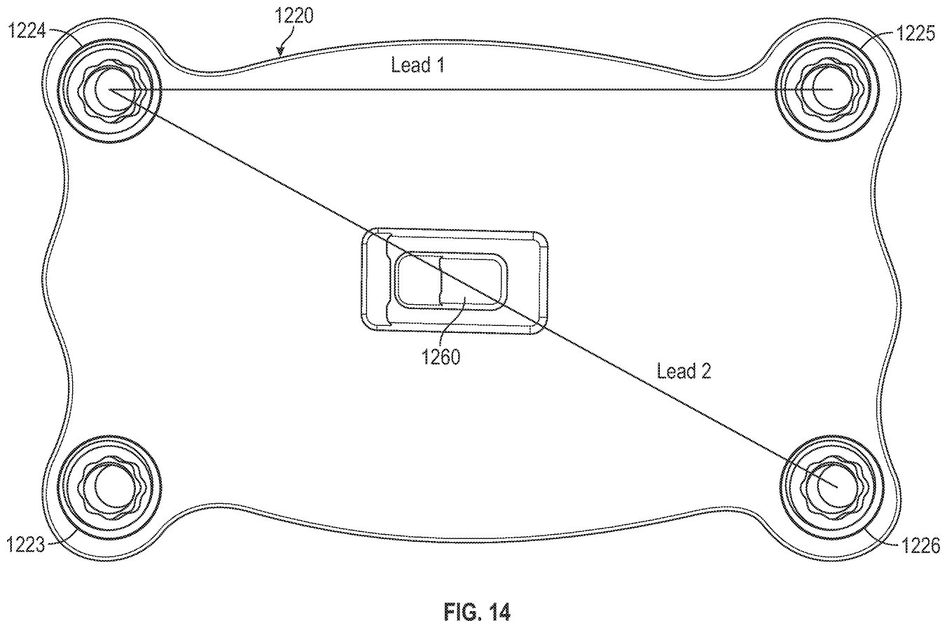

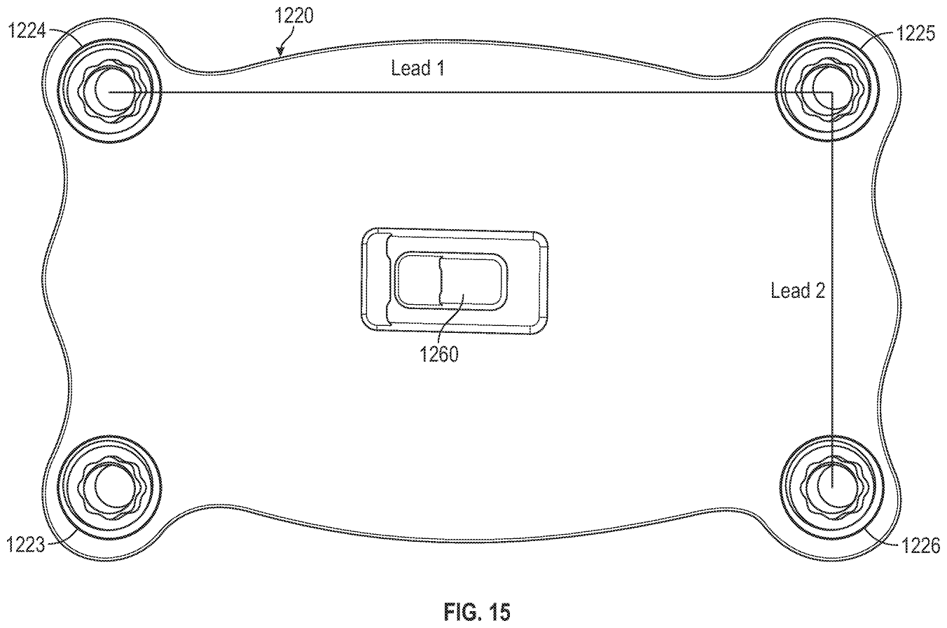

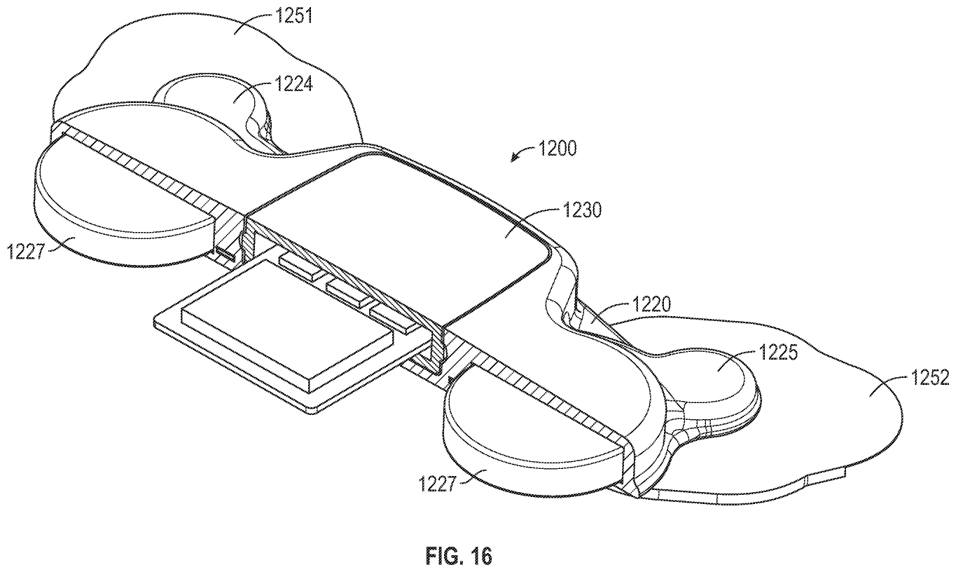

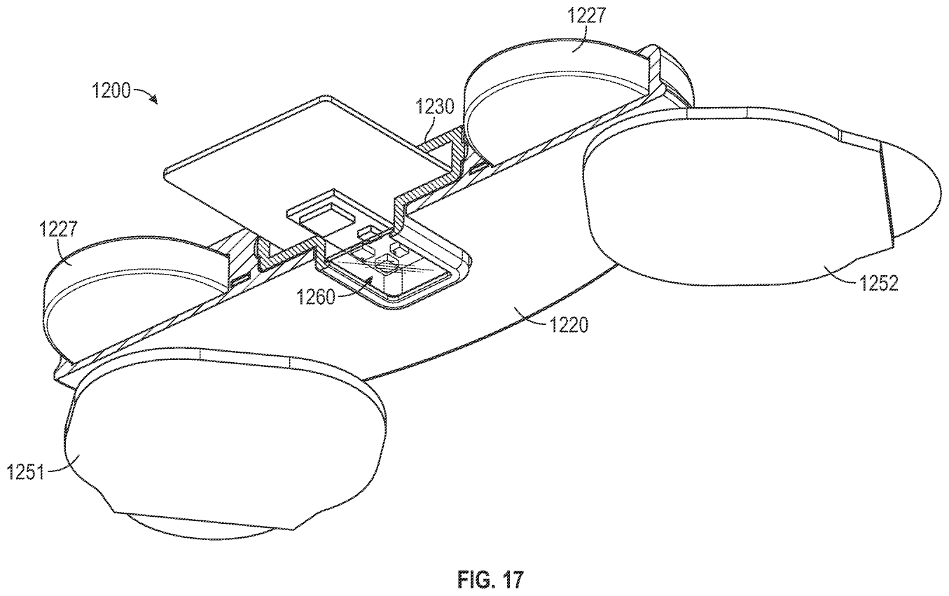

Referring to FIGS. 14 and 15, a bottom view of the platform 1220 of the device 1200 described above is shown, and possible lead connection configurations between three electrodes are illustrated. In the embodiments shown in FIGS. 14 and 15, the connection points 1224, 1225, and 1226 may be electrode connections, creating two lead connections between the three electrodes. Also, referring to FIGS. 16 and 17, perspective cutaway views of the platform 1220 of the device 1200 described above are shown.

Some embodiments of the disclosure may comprise a biosensor device comprising a frame configured to contain (entirely) leads for electrode patches, with attachment points for electrode patches (such that the leads allow signals detected by electrode patches attached to the attachment points to transmit to a processing unit and/or a processing unit attachment point), to space the patches apart for use in medical monitoring (e.g., ECG monitoring) (e.g., the electrode patch points of attachment are so spaced), and to contain one or more batteries and to electrically connect such batteries to the processing unit (e.g., battery terminal(s) electrically connected to the processing unit).

In some embodiments, the device may further comprise a processing unit configured to detect, gather, and/or transmit detected patient data received from the electrodes/leads. In some embodiments, the processing unit is removably attached to the frame (e.g., at a device interface/processing unit attachment point on the frame). In some embodiments, the batteries are not housed within the processing unit and/or permanently attached to the processing unit. In some embodiments, the device may further comprise one or more batteries. In some embodiments, the processing unit further comprises a low energy and/or short-range wireless transmitter (e.g., Bluetooth). In some embodiments, the batteries are not rechargeable and/or lithium-ion (e.g., the batteries may be high capacity cell batteries (such as watch batteries)) or may be rechargeable and/or lithium-ion as technology improves. In some embodiments, the one or more batteries are configured to power the device (and/or the device is configured to use sufficiently low power so that the batteries can power the device) for continuous complete data collection and transmission (e.g., beat-by-beat ECG) monitoring for at least 30 days (e.g., 30-50 days) without recharging and/or changing of batteries. In some embodiments, the device is configured for use with one, two or more standard electrode patches or a proprietary patch.

In some embodiments, the device may further comprise one, two or more standard patches or a proprietary patch attached to the patch-lead attachment points. In some embodiments, the patch-lead attachment points are configured to allow for removable attachment of the patches to the leads. In some embodiments, the frame further comprises patch-lead attachment points, wherein the leads electrically connect the patch-lead attachment points to the processing unit and/or processing unit attachment point/device interface (e.g., for removable processing unit) and/or batteries, and/or wherein the leads are entirely contained within the frame. In some embodiments, the frame comprises battery attachment points (e.g., terminals) configured to allow the batteries to power the processing unit and/or the electrode patch detection of patient data, and/or the batteries are entirely housed within or on the frame (and/or are external to the processing unit). In some embodiments, the batteries are mounted on the patches, and the patch-lead attachment points are configured to transmit both detected ECG data of the patient and power from the batteries to the processing unit. In some embodiments, the frame/housing is waterproof (e.g., allowing a patient to wear the device in the shower and/or in a rain storm) and/or lightweight and/or has no sharp angles on its exterior surface (e.g., ovaloid in shape, like a flat oval). In some embodiments, the device is configured for auto activation and deactivation based on attachment/wearing by patient (e.g., wherein there is no on-off switch, but the processing unit automatically activates and remains active when and for the duration of the electrode patches being attached to a patient's skin to complete the circuit).

Some embodiments of the device may comprise a system comprising the device described above, a smart (cell) phone or other mobile device (e.g., configured to receive detected patient data from the processing unit, for example via Bluetooth) and to transmit (e.g., via Wi-Fi and/or cellular service3) the data to cloud-based processing, a database, a doctor's office, etc. (e.g., for off-site analysis and/or processing). In some embodiments, the cloud, etc. provides instantaneous analysis/processing (e.g., via algorithm) of the detected patient data (such as ECG beat-to-beat data).

Some embodiments of the disclosure may comprise a method of using a device or system (such as those described above), comprising one or more of the following steps: (removably) attaching standard electrode patches to the frame; (removably) attaching the processing unit to the frame; removably attaching the biosensor device to the patient's skin (e.g., by attaching the frame to the patient (solely) using the patches); detecting, collecting, and or transmitting (e.g., by the processing unit) all detected patient data (e.g., continuous beat-by-beat ECG monitoring for at least 30 days (e.g., 30-50 days)) without recharging and/or changing of batteries; transmitting (e.g., by the processing unit) the detected data via Bluetooth to a mobile device (such as a smart phone) located in proximity to the biosensor device (e.g., without removing the processing unit from the frame); retransmitting (e.g., by the mobile device) the detected data via longer-range wireless to cloud processing, remote medical database, doctor office, etc. (e.g., for instantaneous analysis, for example by automated algorithm); analyzing all detected data remotely (e.g., by comparing the detected ECG data to one or more profiles indicative of various medical issues/alert); transmitting (by the cloud, etc.) an alert (e.g., to the wireless device) in response to the detected data matching one or more such profiles and/or transmitting (by the wireless device) the alert to the biosensor device; switching/replacing from one type of standard patch to another standard patch in response to a patient's allergic response or other medical reason; and/or switching/replacing patches that become loose; and/or wherein the biosensor device transmits via Bluetooth all detected data (e.g., all detected beat by beat ECG data) for at least 30 (e.g., 30-50) continuous days (e.g., without recharging or replacing batteries); and/or wherein no algorithm is used by the processing unit to analyze the data or to use or transmit less than all of the detected data; and/or wherein no patient data is stored locally at the device/processing unit; and/or wherein no loose lead wire (e.g., all lead wires completely contained within the frame).

In some embodiments, the device may further comprise one or more data channels (e.g., 1-12 channels of ECG data).

Having described various devices and methods herein, exemplary embodiments or aspects can include, but are not limited to:

Embodiment No. 1 is a biosensor device comprising a frame configured to contain leads for electrode patches, comprising attachment points for electrode patches configured to allow signals detected by electrode patches attached to the attachment points to transmit to a processing unit, and configured to space the patches apart for use in medical monitoring, wherein the frame is also configured to contain one or more batteries and to electrically connect such batteries to the processing unit.

Embodiment No. 2 the device of Embodiment No. 1, further comprising a processing unit configured to detect, gather, and transmit detected patient data received from the electrodes.

Embodiment No. 3 the device of Embodiment No. 2, wherein the processing unit is removably attached to the frame at processing unit attachment point on the frame.

Embodiment No. 4 the device of one of Embodiment Nos. 2-3, wherein the batteries are not housed within the processing unit and are not permanently attached to the processing unit.

Embodiment No. 5 the device of one of Embodiment Nos. 1-4, further comprising one or more batteries configured to be held within the frame.

Embodiment No. 6 the device of one of Embodiment Nos. 1-5, wherein the processing unit further comprises a low energy, short range wireless transmitter.

Embodiment No. 7 is the device of one of Embodiment Nos. 1-6, wherein the batteries are not rechargeable and/or lithium-ion.

Embodiment No. 8 the device of one of Embodiment Nos. 1-7, wherein the one or more batteries are configured to power the device for continuous complete data collection and transmission monitoring for at least 30 days without recharging or changing of batteries.

Embodiment No. 9 the device of one of Embodiment Nos. 1-8, wherein the device is configured for use with one, two or more standard electrode patches.

Embodiment No. 10 the device of one of Embodiment Nos. 1-9, further comprising at least one standard patch attached to the patch-lead attachment points of the frame.

Embodiment No. 11 is the device of Embodiment No. 10, wherein the patch-lead attachment points are configured to allow for removable attachment of the patches to the leads.

Embodiment No. 12 is the device of Embodiment Nos. 1-11, wherein the frame further comprises patch-lead attachment points, wherein the leads electrically connect the patch-lead attachment points to the processing unit and batteries, and wherein the leads are entirely contained within the frame.

Embodiment No. 13 is the device of Embodiment Nos. 1-12, wherein the frame comprises battery attachment points configured to allow the batteries to power the processing unit and/or the electrode patch detection of patient data, and/or the batteries are entirely housed within or on the frame.

Embodiment No. 14 is the device of Embodiment Nos. 1-13, wherein the batteries are mounted on the patches, and wherein the patch-lead attachment points are configured to transmit both detected ECG data of the patient and power from the batteries to the processing unit.

Embodiment No. 15 is the device of one of Embodiment Nos. 1-14, wherein the frame is waterproof and lightweight.

Embodiment No. 16 is the device of one of Embodiment Nos. 1-15, wherein the device is configured for auto activation and deactivation based on attachment by the patient.

Embodiment No. 17 is a communication system comprising a biosensor device comprising a frame configured to contain leads for electrode patches, comprising attachment points for electrode patches configured to allow signals detected by electrode patches attached to the attachment points to transmit to a processing unit, and configured to space the patches apart for use in medical monitoring, wherein the frame is also configured to contain one or more batteries and to electrically connect such batteries to the processing unit; and a smart phone or other mobile device configured to wirelessly receive data from the biosensor device and configured to transmit the received data to at least one of a cloud-based processing system, a database, and a healthcare provider's office.

Embodiment No. 18 is the system of Embodiment No. 17, wherein at least one of the cloud-based processing system, the database, and the healthcare provider's office provides instantaneous analysis of the detected patient data.

Embodiment No. 19 is a method of using a biosensor device comprising attaching one or more standard electrode patches to a frame of the biosensor device; attaching a processing unit to the frame, wherein the processing unit is configured to receive data from the one or more standard electrode patches; removably attaching the biosensor device to the patient's skin via the one or more standard electrode patches; continuously detecting patient data via the one or more standard electrode patches; transmitting, by the processing unit, the detected data via short-range wireless communication to a mobile device located in proximity to the biosensor device; retransmitting, by the mobile device, the detected data via a longer-range wireless to at least one of a cloud processing system, a remote medical database, a healthcare provider office; analyzing all detected data remotely from the biosensor device by comparing the detected data to one or more profiles indicative of various alerts; and transmitting an alert to at least one of the wireless device and the biosensor device in response to the detected data matching one or more such profiles.

Embodiment No. 20 is the method of Embodiment No. 19, wherein the biosensor device transmits via Bluetooth all detected data for at least 30 continuous days without recharging or replacing batteries.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above but is defined by the claims which follow that scope including all equivalents of the subject matter of the claims. Each and every claim is incorporated as further disclosure into the specification and the claims are embodiment(s) of the present presently disclosed subject matter. Furthermore, any advantages and features described above may relate to specific embodiments but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the subject matter set out in any claims that may issue from this disclosure. Specifically, and by way of example, although the headings might refer to a “Field,” the claims should not be limited by the language chosen under this heading to describe the so-called field. Further, a description of a technology in the “Background” is not to be construed as an admission that certain technology is prior art to any subject matter of this disclosure. Neither is the “Summary” to be considered as a limiting characterization of the subject matter set forth in issued claims. Any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure but should not be constrained by the headings set forth herein.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed devices, systems, and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A biosensor device comprising:
a frame configured to contain leads for electrode patches, comprising attachment points for electrode patches configured to allow signals detected by electrode patches attached to the attachment points to transmit to a processing unit, and configured to space the patches apart for use in medical monitoring, wherein the frame is also configured to contain one or more batteries and to electrically connect such batteries to the processing unit.

2. The device of claim 1, further comprising a processing unit configured to detect, gather, and transmit detected patient data received from the electrodes.

3. The device of claim 2, wherein the processing unit is removably attached to the frame at processing unit attachment point on the frame.

4. The device of claim 2, wherein the batteries are not housed within the processing unit and are not permanently attached to the processing unit.

5. The device of claim 1, further comprising one or more batteries configured to be held within the frame.

6. The device of claim 1, wherein the processing unit further comprises a low energy, short range wireless transmitter.

7. The device of claim 1, wherein the batteries are not rechargeable and/or lithium-ion.

8. The device of claim 1, wherein the one or more batteries are configured to power the device for continuous complete data collection and transmission monitoring for at least 30 days without recharging or changing of batteries.

9. The device of claim 1, wherein the device is configured for use with one, two or more standard electrode patches.

10. The device of claim 1, further comprising at least one standard patch attached to the patch-lead attachment points of the frame.

11. The device of claim 10, wherein the patch-lead attachment points are configured to allow for removable attachment of the patches to the leads.

12. The device of claim 1, wherein the frame further comprises patch-lead attachment points, wherein the leads electrically connect the patch-lead attachment points to the processing unit and batteries, and wherein the leads are entirely contained within the frame.

13. The device of claim 1, wherein the frame comprises battery attachment points configured to allow the batteries to power the processing unit and/or the electrode patch detection of patient data, and/or the batteries are entirely housed within or on the frame.

14. The device of claim 1, wherein the batteries are mounted on the patches, and wherein the patch-lead attachment points are configured to transmit both detected ECG data of the patient and power from the batteries to the processing unit.

15. The device of claim 1, wherein the frame is waterproof and lightweight.

16. The device of claim 1, wherein the device is configured for auto activation and deactivation based on attachment by the patient.

17. A communication system comprising:
a biosensor device comprising a frame configured to contain leads for electrode patches, comprising attachment points for electrode patches configured to allow signals detected by electrode patches attached to the attachment points to transmit to a processing unit, and configured to space the patches apart for use in medical monitoring, wherein the frame is also configured to contain one or more batteries and to electrically connect such batteries to the processing unit; and
a smart phone or other mobile device configured to wirelessly receive data from the biosensor device and configured to transmit the received data to at least one of a cloud-based processing system, a database, and a healthcare provider's office.

18. The system of claim 17, wherein at least one of the cloud-based processing system, the database, and the healthcare provider's office provides instantaneous analysis of the detected patient data.

19. A method of using a biosensor device comprising:
attaching one or more standard electrode patches to a frame of the biosensor device;
attaching a processing unit to the frame, wherein the processing unit is configured to receive data from the one or more standard electrode patches;
removably attaching the biosensor device to the patient's skin via the one or more standard electrode patches;
continuously detecting patient data via the one or more standard electrode patches;
transmitting, by the processing unit, the detected data via short-range wireless communication to a mobile device located in proximity to the biosensor device;
retransmitting, by the mobile device, the detected data via a longer-range wireless to at least one of a cloud processing system, a remote medical database, a healthcare provider office;
analyzing all detected data remotely from the biosensor device by comparing the detected data to one or more profiles indicative of various alerts; and
transmitting an alert to at least one of the wireless device and the biosensor device in response to the detected data matching one or more such profiles.

20. The method of claim 19, wherein the biosensor device transmits via Bluetooth all detected data for at least 30 continuous days without recharging or replacing batteries.
